(19) United States
(12) Patent Application Publication
(10) Pub. No.: US 2006/0106026 A1
(43) Pub. Date: May 18, 2006

(54) 4-(4-methylpiperazin-1-ylmethyl)-n-[4-pyridin-3-yl)pyrimidin-2-ylamino)phenyl]-benzamide for treating anaplastic thyroid cancer

(76) Inventors: Akira Ohtsuru, Nagasaki (JP); Alexej Podtcheko, Nagasaki (JP); Satoshi Tsuda, Nagasaki (JP); Shunichi Yamashita, Nagasaki (JP)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/521,929

(22) PCT Filed: May 23, 2003
(86) PCT No.: PCT/IB03/01985

§ 371 Date: Aug. 5, 2005

Related U.S. Application Data
(60) Provisional application No. 60/398,410, filed on Jul. 24, 2002.
(60) Provisional application No. 60/454,557, filed on Mar. 14, 2003.

(51) Int. Cl. A61K 31/506

(52) U.S. Cl. 514/252.18

(57) ABSTRACT

The invention relates to the use of 4-(4-methylpiperazin-1-ylmethyl)-N-[4-methyl-3-(4-pyridin-3-yl)pyrimidin-2-ylamino)phenyl]-benzamide of the following formula 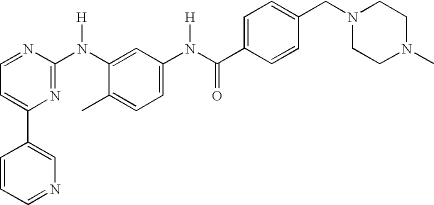 or a pharmaceutically acceptable salt thereof for the manufacture of a medicament for the treatment of anaplastic thyroid cancers

The invention relates to the use of 4(4-methylpiperazin-1-ylmethyl)N-[4-methyl-3-(4pyridin-3-yl)pyrimidin-2-ylamino)phenyl]-benzamide (hereinafter: “COMPOUND I”) or a pharmaceutically acceptable salt thereof for the manufacture of a medicament for the treatment of anaplastic thyroid cancers, to the use of COMPOUND I or a pharmaceutically acceptable salt thereof in the treatment of anaplastic thyroid cancer, to a method of treating warm-blooded animals including mammals, especially humans suffering from anaplastic thyroid cancer by administering to a said animal in need of such treatment an effective dose of COMPOUND I or a pharmaceutically acceptable salt thereof.

Thyroid follicular cell-derived carcinomas are classified pathologically as differentiated (papillary and follicular) and undifferentiated (anaplastic) carcinomas. Differentiated carcinomas have relatively good prognosis, however anaplastic thyroid carcinomas are highly aggressive and extremely lethal, with poor therapeutic response. The prevalence of tumor suppressor p53 gene mutations in anaplastic carcinomas has been reported at 70-85% vs. 0-9% in differentiated carcinomas. The mutations in the p53 gene are therefore recognized as a late genetic event associated with loss of differentiation in thyroid carcinogenesis and one of the molecular changes responsible for the highly aggressive property of this type of carcinoma.

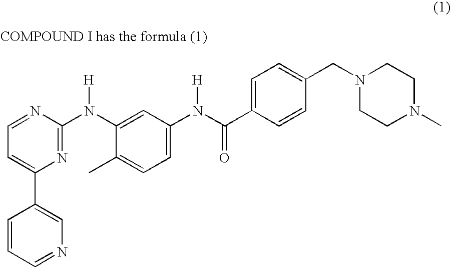

COMPOUND I free base and its acceptable salts thereof are disclosed in the European Patent application 0564409.

Pharmaceutically acceptable salts of COMPOUND I are pharmaceutically acceptable acid addition salts, like for example with inorganic acids, such as hydrochloric acid, sulfuric acid or a phosphoric acid, or with suitable organic carboxylic or sulfonic acids, for example aliphatic mono- or di-carboxylic acids, such as trifluoroacetic acid, acetic acid, propionic acid, glycolic acid, succinic acid, maleic acid, fumaric acid, hydroxymaleic acid, malic acid, tartaric acid, citric acid or oxalic acid, or amino acids such as arginine or lysine, aromatic carboxylic acids, such as benzoic acid, 2-phenoxy-benzoic acid, 2-acetoxy-benzoic acid, salicylic acid, 4-aminosalicylic acid, aromatic-aliphatic carboxylic acids, such as mandelic acid or cinnamic acid, heteroaromatic carboxylic acids, such as nicotinic acid or isonicotinic acid, aliphatic sulfonic acids, such as methane-, ethane- or 2-hydroxy-ethane-sulfonic acid, or aromatic sulfonic acids, for example benzene-, p-toluene- or naphthalene-2-sulfonic acid.

COMPOUND I mesylate, herein after denominated “SALT I” and COMPOUND I mesylate alpha and beta crystal forms are disclosed in International Patent application WO 99/03854 published on January 1999.

Surprisingly, it has now been found that SALT I can be used as a therapeutic agent for the treatment of anaplastic thyroid carcinomas, especially in anaplastic thyroid carcinomas harboring mutated p53.

Hence, the invention relates to a method of treating a warm-blooded animal having anaplastic thyroid carcinoma comprising administering to said animal in need of such a treatment SALT I in a quantity which is therapeutically effective against anaplastic thyroid carcinomas, especially in anaplastic thyroid carcinomas harboring mutated p53.

The invention relates to a method for administering to a human subject suffering from anaplastic thyroid carcinomas, especially in anaplastic thyroid carcinomas harboring mutated p53, an acid addition salt and preferably the monomethanesulfonate salt of 4-(4-methylpiperazin-1-ylmethyl)-N-[4-methyl-3-(4-pyridin-3-yl)pyrimidin-2-ylamino)phenyl]-benzamide of the formula I.

The term “treatment” comprises the administration of SALT I to a warm-blooded animal in need of such treatment with the aim to cure the tumor or to have an effect on tumor regression or on the delay of progression of a disease.

The term “delay of progression” as used herein means that the tumor growth or generally, the disease progression is at least slowed down or hampered by the treatment and that patients exhibit higher survival rates than patients not being treated or being treated with placebo.

The pharmaceutical compositions according to the present invention can be prepared in a manner known per se and are those suitable for enteral, such as oral or rectal, and parenteral administration to warm-blooded animals, including man, comprising a therapeutically effective amount of at least one pharmacologically active ingredient, alone or in combination with one or more pharmaceutically acceptable carries, especially suitable for enteral or parenteral application. The preferred route of administration of the dosage forms of the present invention is orally.

The person skilled in the pertinent art is fully enabled to select relevant test models to prove the beneficial effects mentioned herein on anaplastic thyroid carcinomas. The pharmacological activity of such a compound may, for example, be demonstrated by means of the Examples described below, by in vitro tests and in vivo tests in nude or transgenic mice or in suitable clinical studies. Suitable clinical studies are, for example, open label non-randomized, dose escalation studies in patients with anaplastic thyroid carcinomas. The efficacy of the treatment is determined in these studies, e.g., by evaluation of the carcinoma's size every 6 weeks or by suitable serum tumor markers or by scintigraphy tumor detection with the control achieved on placebo matching with the active ingredient.

The effective dosage of SALT I may vary depending on the particular compound or pharmaceutical composition employed, on the mode of administration, the type of the thyroid cancer being treated or the severity of the thyroid cancer being treated. The dosage regimen is selected in accordance with a variety of further factors including the renal and hepatic function of the patient. A physician, clinician or veterinarian of ordinary skill can readily determine and prescribe the effective amount of compounds required to prevent, counter or arrest the progress of the condition.

Depending on species, age, individual condition, mode of administration, and the clinical picture in question, effective doses of SALT I, for example daily doses corresponding to about 10-1000 mg of the active compound (free base), preferably 50-600 mg, especially 100 to 400 mg, are administered to warm-blooded animals of about 70 kg body weight. For adult patients with anaplastic thyroid cancer, especially in anaplastic thyroid carcinomas harboring mutated p53, a starting dose of 200 or 400 mg daily can be recommended. For patients with an inadequate response after an assessment of response to therapy, dose escalation can be safely considered and patients may be treated as long as they benefit from treatment and in the absence of limiting toxicities.

The present invention relates also to a method for administering to a human subject suffering from anaplastic thyroid cancer, especially in anaplastic thyroid carcinomas harboring mutated p53, COMPOUND I or a pharmaceutically acceptable salt thereof which comprises administering a pharmaceutically effective amount of COMPOUND I or a pharmaceutically acceptable salt thereof to the human subject, e.g., once daily, e.g. for a period exceeding 3 months. The invention relates especially to such method wherein a daily dose of 50 to 600 mg, preferably 100 to 400 mg is administered to an adult

### EXAMPLE 1

### SALT I Induces S-G2 Transition Cell Arrest in Anaplastic Thyroid Cancer Cells

1) Suppressive Effect of SALT I on Cell Growth

Cell Growth Assays—Experiment 1:

Cell Culture: Human anaplastic or undifferentiated thyroid carcinoma cell lines, FRO and ARO, with undetectable or mutant p53 and differentiated papillary thyroid carcinoma with wild type p53 gene (KTC-1) are prepared. 1F3 cell line is a stable transformant with introduction of wild type p53 gene to FRO. These cell lines are cultured in RPMI 1640 medium supplemented with 5% heat-inactivated fetal bovine serum (FBS; Life Technologies, Inc.) and are maintained at 37° C. in a humidified atmosphere of 5% CO₂. For analysis of the effect of SALT I, cells are incubated in the presence of 0.1% dimethyl sulfoxide (DMSO) or SALT I which is diluted by DMSO (final concentration 0.1%). Cells are seeded at a density of 1×10³ cells/well in a 96-wellmicrotiter plate (n=5). One day later (day 1), cells are treated with 1, 10, or 50 μM of SALT I or 0.1% DMSO in 100 μl of fresh medium. Cell number of each well is measured with a cell count kit (Wako) after a 48 hour-incubation. This experiment is performed at least three times.

Cell growth curve is examined using cytometer as below. Cells are seeded at a density of 0.5 or 0.8×10⁵ cells/well in a 6-well-culture plate (n=5). One day later (day 1), cells are incubated in the presence of 10 μM of SALT I or 0.1% DMSO. Cell number is counted at day 2, 3, 4, and 5. This experiment is performed at least three times.

Results: After a 48 hour-treatment with SALT I at concentrations ranging from 0 to 50 μM, cell number is quantified with a cell count kit (Wako). The values of the table represent the mean of 5 independent experiments.

| | cell lines | | | | | |
|---|---|---|---|---|---|---|
| | FRO | | ARO | | KTC-1 | |
| | mean | SD | mean | SD | mean | SD |
| no SALT I | 1.677 | 0.020 | 0.959 | 0.149 | 0.413 | 0.028 |
| SALT I 1 μM | 1.414 | 0.131 | 0.785 | 0.032 | 0.403 | 0.028 |
| SALT I 10 μM | 1.192 | 0.053 | 0.539 | 0.071 | 0.379 | 0.040 |
| SALT I 50 μM | 0.228 | 0.013 | 0.096 | 0.015 | 0.064 | 0.016 |

Table I: All cell lines died at a concentration 50 μM of SALT I. However, undetectable (FRO) or mutant p53 (ARO) cell lines show growth suppression in SALT I dose dependent manner lower than 50 μM, but wild type p53 (KTC-1) cell line do not

Cell growth curve on a 5 day period show time-dependent reduction of cell growth in FRO and ARO cells treated with 10 μM of SALT I compared to control cells. SALT I do not change the cell growth of KTC-1 and IF3 cells (data not shown).

Cell Growth Assays—Experiment 2:

Human anaplastic thyroid carcinoma cell lines FRO and ARO were used with respectively, undetectable or mutant p53 in codon 273 (Fagin et al., J. Clin. Invest. 1993, 91:179-184). Human papillary carcinoma cell line NPA has p53 mutations in codons 223 and 226 (Fagin et al., J. Clin. Invest. 1993, 91:179-184), while TPC-1 and KTC-1 papillary thyroid carcinoma cell lines are wild type for p53 (Kurebayashi et al., J. Clin. Endocrinol. Metab. 2000, 85:2889-96). Stable transfection with a vector expressing wild type p53 was used to produce the 1F3 cell line from FRO cells (Zeki et al., Int. J. Cancer, 1998, 75:391-5). Cells were cultured in RPMI 1640 medium supplemented with 5% FBS at 37° C. in a humidified atmosphere with 5% CO₂. Primary human thyroid cell cultures were established as described previously (Kawabe et al., J. Clin. Endocrinol. Metab., 1989, 68:1174-83) and maintained in a 2:1 mixture of F12 Nutrient Mixture and DEM, supplemented with 3% fetal bovine serum and penicillin-streptomycin (all reagents from Invitrogen Life Technologies, Paisley, UK).

Cells are seeded at a density of 1×10³ cells/well in a 96-well microtiter plate. One day later (day 1), cells are treated with 1, 5, 10, 20 or 50 μM of SALT I diluted in DMSO or 0.1% DMSO in 100 μl of fresh medium (6 wells fro each drug concentration). Cell number of each well is measured with a cell count kit (Wako) after a 72 h of incubation. IC₅₀ values, defined as the concentrations of SALT I producing a 50% reduction in cell growth, were estimated by linear interpolation at r=0.5. The kinetics of cell growth were examined using a cytometer as follows: cells were seeded at a density of 0.5 or 0.1×10⁵ cells per well in 12-well culture plates. One day later (day 1), they were given medium containing 10 μM SALT I or DMSO 0.1% and counted on days 2,3,4 and 5. Both experiments were performed at least three times.

| | | Cell lines | | | | | |
|---|---|---|---|---|---|---|---|
| | | ARO | FRO | NPA | TPC-1 | KTC-1 | PT |
| IC₅₀ values | mean | 8.1* | 6.4* | 15.6 | 29.5 | 22.6 | 24.7 |
| in μM | SD | 0.8 | 0.6 | 1.3 | 2.6 | 2.1 | 2.3 |
| | n | 3 | 3 | 3 | 3 | 3 | 3 |

Table 2: Effect of SALT I on the growth of human thyroid cancer cell lines. IC₅₀ values for the effect of SALT I on growth rate.

The effect of SALT I on five thyroid cancer cell lines, and on primary cultures of human thyrocytes, was measured by means of the standard WST-assay. As shown in Table 2, the IC₅₀ for the p53-mutant cell lines FRO, ARO and NPA was significantly lower than for the wild type p53 cell lines TPC-1 and KTC-1, and for primary thyrocytes (PT). SALT I selectively suppresses the growth of anaplastic thyroid cell lines. Data are representative of at least three separate experiments; each value combines the results of 6 wells. *, P<0.05 comparing control vs. treatment.

2) Expression of c-Abl, PDGF Receptor and c-kit

SALT I inhibits the tyrosine kinase activity of c-Abl, PDGF receptor and c-kit. Reverse transcription-polymerase chain reaction (RT-PCR) on total RNA extracted from thyroid carcinoma cell lines shows that the PDGF receptor and c-kit are not expressed in FRO and ARO cells (data not shown), however all cell lines (ARO, FRO, TPC, KTC, NPA and PT) express the c-Abl mRNA.

3) Inhibition of S-G2 Transition by SALT I

Sub-confluent cells were incubated for 48 hours with 10 μM of SALT I or 0.1% DMSO. For flow cytometry analysis, cells were fixed with 70% ethanol and wash with PBS. After pre-incubation with RNase A (0.1 mg per ml) at room temperature, cells were stained with PI (25 μg per ml). Fluorescence was measured by using FACScan flow cytometer (Becton Dickinson, Mountain View, Calif.). This experiment was performed at least three times.

| | G1 | | S | | G2-M | |
|---|---|---|---|---|---|---|
| | DMSO | SALT I | DMSO | SALT I | DMSO | SALT I |
| FRO | 32.16 | 30.91 | 43.26 | 54.11 | 24.95 | 14.98 |
| KTC-1 | 72.2 | 74.77 | 7.68 | 6.53 | 20.11 | 18.7 |
| 1F3 | 63.3 | 70.5 | 9.4 | 9.1 | 27.3 | 20.4 |
| ARO | 76.6 | 77.6 | 19.7 | 13.1 | 3.8 | 9.3 |
| TPC-1 | 67.8 | 77.6 | 9.7 | 9.3 | 22.6 | 13.1 |

Table 3: Effect of SALT I on the cell cycle in thyroid cancer cell lines. Cells were treated with 0.1% DMSO only or with 10 μM of SALT I for 48 hours and analyzed for cell cycle distribution by flow cytometry (results are given as percentage of cell in G2, S and G2-M phase).

The FACS analysis of cell cycle showed that SALT I treatment increased S phase (43% vs. 54%) and decreased G2-M phase (25% vs. 15%) in FRO cells, but no alteration observed in KTC-1 cells or in 1F3 cells. This result indicated that SALT I induced S-G2 transition cell arrest in FRO cells. FACS cell cycle analysis showed that treatment with SALT I increased more then two-fold the proportion of cells in G2/M-phase (9.28% vs. 3.78%) in the ARO cell line and elevated the number of cells in S-phase (54% vs. 43%) in FRO. No such changes were observed in TPC-1, KTC-1 and 1F3 cells. In those cell lines, there was a tendency for the proportion of cells in G1-phase to increase. However, no growth inhibition was observed in the cultures treated with SALT I. Thus, the treatment with SALT I causes G2/M- and S-phase arrest in ARO and FRO cells, respectively, and that this may be the cause of the observed growth inhibition in these cell lines. The apoptotic fraction (sub-G1) did not significantly increase after 48 hours of treatment with the drug in any cell line. Furthermore, no DNA fragmentation was detected in the DNA ladder assay after 48 hours of treatment (data not shown).

4) Western Blotting Analysis: Effect of SALT I on Cell Cycle Regulatory Proteins in Thyroid Cancer Cell Lines.

Cells were treated with 10 μM of SALT I for 0, 12, 24, 48 hours and cell lysates were prepared in RIPA buffer and resolved by SDS-PAGE (40 μg proteins/lane). After transfer onto nitrocellulose membranes (Pall Corporation, Ann Arbor, Mich., USA), blots were probed with the appropriate antibodies. β-actin was used as a loading control. The antibodies used were: anti-p21waf1 (Ab-1, Calbiochem, Darmstardt, Germany), anti-p27-8, Santa Cruz Biotechnology, Santa Cruz, Calif., USA), anti-cyclin A (C88020, BD Biosciences, Boston, Mass., USA), anti-cyclin B1 (C23420, BD Biosciences), anti-CDK1/Cdc2 (C12720, BD Biosciences), anti-cyclin D3 (C28620, BD Biosciences), anti-Phospho-c-ABL (Tyr245, Cell Signaling Technology, Beverly, Mass., USA), anti-c-Abl (24-11, Santa Cruz Biotechnology), anti-c-KIT (C-14, Santa Cruz Biotechnology), anti-PDGFRα (C-20, Santa Cruz Biotechnology), anti-PDGFRβ (P-20, Santa Cruz Biotechnology), anti-ERK1/2 (Cell Signalling Technology), anti-p-ERK (Cell Signalling Technology) and anti-Actin (C-11, Santa Cruz Biotechnology). Detection was performed with an enhanced chemiluminescence kit (ECL, Amersham Life Sciences, Buckinghamshire, UK). Immunoblotting experiments were performed at least twice. Results: Western blotting analysis detected high level of c-Abl protein in the anaplastic thyroid cancer cell lines FRO and ARO and in the p53-mutant papillary cancer cell line NPA (data not shown). Protein bands corresponding to other SALT I-sensitive tyrosine kinases were not detected in these cell lines. Since p53 status is likely to have some effect on the level of c-Abl protein, the expression level of c-Abl in 1F3 cells was also measured. 1F3 cells contain less c-Abl than FRO cells but more than normal thyrocytes. The cyclin-dependent kinase (CDK) inhibitors, p21^{cip1} and p27^{kip1}, can block CDK activity in the S to G2 as well as in the G1 to S phase transition of the cell cycle. Expression of p2^{cip1} in FRO cells was markedly increased after 12 hours of exposure to SALT I, but did not change in ARO, KTC-1 and 1F3 cells. Expression of p27^{kip1} increased in ARO and FRO cells after 24 and 48 hours of exposure, respectively. The activity of CDKs is dependent, in part, on the relative abundance of cyclin subunits and the presence of CDK inhibitors. Among the cyclins and CDKs, cyclin A, B, and CDC2 are involved in the progression from G2 to M phase. 24 hours of SALT I treatment reduced the levels of cyclin A, B1 and CDC2 in the ARO and FRO cell lines and of cyclin D3 in ARO cells, but had no effect in KTC-1 and 1F3. Under same conditions levels of β-actin were not significantly affected.

5) In Vitro Kinase Assay: Phosphorylation of c-Abl and MAPK Kinase Activity

Abl was immunoprecipitated from cell lysates using the indicated antibody. In vitro kinase assay was performed as described previously (Dorey et al., Oncogene, 2000, 56:8075-84). Radiolabelled GST-Crk was quantified using a PhosphorImager (Molecular Dynamics, Inc., Sunnyvale, Calif., USA). The cells were treated with various doses of SALT I for 12 hours, and cell extracts were subjected to Western blot analysis with antibodies to phosphorylated and total c-Abl. c-Abl activity was determined by in vitro kinase assay with ³²P-ATP using GST-Crk as substrate. For MAPK kinase activity, cells were incubated in serum free medium with 0.1% DMSO or 10 μM SALT I for two hours. Stimulation was with 20% FCS for 8 minutes, cell lysates were collected and subjected to SDS-PAGE and Western blotting carried out using antibodies against ERK1/2 and the phosphorylated form of ERK1/2. All experiments were performed twice and gave similar results. Results: ARO and FRO cells cultured in normal conditions have high levels of c-Abl and of its Tyr245 phosphorylated form which has been previously associated with significant activation of the c-Abl kinase activity (data not shown). SALT I in concentrations up to 50 μM did not appreciably affect the level of c-Abl protein in these cell lines over the time interval examined. However, 12 hours of continuous treatment did decrease the tyrosine phosphorylation of c-Abl . The inhibition of c-Abl kinase activity, assayed with GST-Crk fusion protein, as a result of treatment of ARO and FRO with SALT I was correlated with the level of c-Abl phosphorylation. In contrast SALT I induced accumulation of c-Abl in wt-p53 cell lines (IF3 and KTC-1), and did not reduce the level of phospho-c-Abl. The mechanism underlying the accumulation of c-Abl after SALT I treatment of wt-p53 thyroid cell lines needs further elucidation. A common response to extracellular signals such as growth factors is the activation of the mitogen-activated protein (MAP) kinase cascade. To determine whether SALT I inhibits the activity of receptor tyrosine kinases expressed in the cell lines under investigation, the effect of SALT I on the phosphorylation of ERK 1/2 in response to serum stimulation. Stimulation increased the level of p-ERK1/2 in starved KTC-1 cells, but SALT I had no effect on its phosphorylation. Serum stimulation did not significantly modulate the activity of ERK1/2 kinase in anaplastic thyroid cancer cell lines ARO and FRO, and SALT I did not affect the level of p-ERK1/2. In addition, supplementation of the medium with PDGF-BB, the ligand of PDGFRα and −Rβ, did not alter MAP kinase activity, and PDGFRβ was not affected by SALT I treatment in ARO and FRO cells (data not shown). These results indicate that c-Abl is likely to be the only SALT I target kinase active in the anaplastic cancer cell lines used in the present experiments.

6) Immunohistological Analysis of c-Abl and p53

Usage of archived fixed tissue sections for immunohistological studies was approved by ethics committee of the Nagasaki University Hospital. Informed consent was obtained from each individual. Immunohistochemistry was performed as described before (Hermann et al., Int. J. Cancer 2001, 92:805-11). Briefly, 4 μm sections of formalin fixed paraffin embedded tissue were deparaffinized, heat antigen demasked (0.01 mol/l citrate buffer, pH 6.0), and exposed to primary antibodies for 1 hr at room temperature. The following antibodies were used: murine monoclonal anti-p53 antibody, clone DO-7 (DAKO, Copenhagen, Denmark, dilution 1:100) and murine monoclonal anti-c-Abl antibody (24-11, Santa Cruz Biotechnology, dilution 1:200). Bound antibodies were visualized with a biotin-conjugated secondary goat-anti-mouse IgG/IgM F(ab)2 antiserum and peroxidase-conjugated streptavidin (Jackson Immuno Research Laboratories, West Grove, Pa., USA). The slides were examined by two independent observers who were not cognizant of the pathological or clinical data on the cases under investigation. For evaluation of p53 staining, 4 high-power fields (×400) were assessed with regard to the percentage of positively stained tumor cells. Tumors with >10% stained cells were assigned as “strongly positive” and those with ≦10% as “weakly positive”, as previously described in Hermann et al., Int J. Cancer 2001, 92:805-11. The c-Abl immunostaining wag semi-quantified by means of a visual grading system in which staining intensity was categorized as Grade 0, 1+, 2+, or 3+, according to the previously reported criteria (Yanagawa et al., Oral Oncol. 2000, 36:89-94). To simplify the correlation of c-Abl level with the histological features of the thyroid cancers, these groups were further classified into “weakly positive” (Grade 0, Grade 1+) and “strongly positive” (Grade 2+, Grade 3+) groups.

Results: To gain more information about c-Abl and p53 expression in different types of human thyroid tumors, immunohistochemical staining have been carried out for these proteins in different types of surgically resected tumors (data not shown). High expression of c-Abl (combined nuclear and cytoplasmic immunostaining) was detected in ⅚ (83%) anaplastic carcinomas, whereas a significantly smaller proportion, 2/9 (22%) and ⅛ (12%), p=0.041 and p=0.026, by Fisher's exact test, was observed in follicular and papillary carcinomas (Table 4). A low level of expression of c-Abl was observed in the normal tissue surrounding the tumor lesions and in cases of adenomatous goiter. A pattern of nuclear p53 staining was detected in all cases of anaplastic carcinomas (6/6), but in only a small fraction of follicular and papillary cancers: 1/9 and ⅛; p=0.0014 and p=0.005, respectively (Fisher's exact test). Only 1/10 (10%) of the benign thyroid lesions (goiter) examined exhibited weak nuclear p53 expression. Thus, the immunohistochemical findings are in accord with the observed over-expression of c-Abl protein in p53-mutant anaplastic thyroid cancer cell lines.

| | Strongly positive | | | Strongly positive | | |
|---|---|---|---|---|---|---|
| | c-Abl staining | | | p53 staining | | |
| | Number | | | Number | | |
| Type of tumor | of cases | % | P* | of cases | % | P* |
| Anaplastic | 5/6 | 83.3 | — | 6/6 | 100 | — |
| Follicular | 2/9 | 22.2 | 0.041 | 1/9 | 11.1 | 0.0014 |
| Papillar | 1/8 | 12.5 | 0.026 | 1/8 | 12.5 | 0.005 |
| Adenomatous Goiter | 0/10 | 0 | 0.0014 | 0/10 | 0 | <0.001 |

Table 4: c-Abl and p53 detection by immunohostochemistry in anaplastic, follicular and papillary carcinomas and a denomatous goiter. Sections were counterstained with hematoxylin for c-Abl and methyl green for p53. Note the staining pattern: c-Abl: nuclear/cytoplasmic, p53: nuclear in AC (data not shown).

7) In Vivo Effect of SALT I on FRO Cells

Mouse xenograft model. All mice were maintained in the Nagasaki University (Nagasaki, Japan) animal facility and all animal experiments described in this study were conducted in accordance with the principles and procedures outlined in the Guide for the Care and Use of Laboratory Animals of the Nagasaki University School of Medicine. Five million FRO cells suspended in RPMI 1640 were injected s.c. into the flanks of 8-week-old female BalB/c nu/nu mice (Charles-River Japan, Tokyo, Japan). Tumor sizes were measured every other day with calipers and tumor volumes were calculated according to the formula: a²×b×0.4 where a is the smallest diameter and b is the diameter perpendicular to a. After the tumors had reached at least 100 m³, the mice were randomly assigned to experimental or control groups, 5 animals per group. SALT I solution in sterile water ass injected i.p. daily for 2 weeks at a dose of 50 mg/kg. Mice in the control group received injections of pure water. The body weight feeding behavior and motor activity of each animal were monitored as indicators of general health.

Statistical analysis: Data are presented as mean ±SD unless otherwise specified. Student's t-test and Mann Whitney U test were used for comparison between two groups for parametric and nonparametric data, respectively. A p value <0.05 is considered statistically significant. Results: To examine a possible anti-tumor effect of SALT I on thyroid anaplastic cancer in vivo, FRO cells were implanted in athymic mice, and SALT I or vehicle (H₂O) was injected intraperitoneally. As shown in the Table 5, single daily administration of 50 mg/kg SALT I over 14 consecutive days resulted in a strong anti-tumor effect. The body weight and physical activity of the mice exposed to SALT I was not significantly affected.

| | Days | 0 | 2 | 4 | 6 | 8 | 10 | 12 | 14 |
|---|---|---|---|---|---|---|---|---|---|
| Mean ± | H₂O | 100 | 108 ± 9.5 | 120 ± 10.4 | 153 ± 28 | 180 ± 32 | 228 ± 45 | 280 ± 89 | 295 ± 97 |
| SEM | SALT I | 100 | 108 ± 12 | 108 ± 10 | 100 ± 13 | 94 ± 12 | 96 ± 15 | 98 ± 13 | 98 ± 16* |
| | n | 5 | 5 | 5 | 5 | 5 | 5 | 5 | 5 |

Table 5: Antitumor effect of SALT I in FRO cells implanted into athymic mice. Animals from each group (n=5) were treated with i.p. injections of either SALT I or placebo (H₂O). The graph shows the dynamics of tumor growth in mm³ in the experimental and control groups. *, P<0.05 comparing control vs. experimental group.

In the present invention is shown that the specific tyrosine kinase inhibitor, SALT I, is a potential anti-cancer drug against undifferentiated thyroid carcinomas harboring mutated p53. Treatment with SALT I induced remarkable growth inhibition in p53-defective FRO, ARO and NPA cell lines, but not in KTC-1 and TPC-1, which have wild type p53. Similarly, there was no effect of SALT I on FRO cells stably transfected with wild type p53, thus confirming that the effect of SALT I is dependent on p53 status. In the p53-mutated anaplastic cancer cell lines ARO and FRO, a cytostatic effect was observed at concentrations that are clinically achievable (IC₅₀ 5.9 and 7.8 μM, respectively). These IC₅₀ values were lower than in NPA cells (IC₅₀ 16 μM) and in other papillary carcinoma cell lines. The present study was focused on anaplastic cancer cells. Flow cytometry revealed that the growth suppression by SALT I was due to arrest in G2/M or late S-phase in such cell lines.

The cytostatic effect of SALT I has been demonstrated not only in CML, but also in small cell lung cancer characterized by increased activity of PDGFR, and in gastrointestinal stromal tumors that show strong c-KIT tyrosine kinase activation.

RT-PCR analysis revealed the presence of c-Abl mRNA in all cell lines. Expression of PD GFRα, PDGFRβ and c-KIT was undetectable or very low in anaplastic cell lines and exhibited various patterns in other cell lines. Using Western blotting, it was found that the level of c-Abl was significantly higher in the anaplastic thyroid cancer cell lines ARO and FRO compared with primary thyrocytes and papillary carcinoma cell lines. In the p53-mutant papillary cancer cell line NPA, the c-Abl level was also higher than in the wt-p53 papillary cancer cell lines TPC-1 and KTC-1. Moreover, stable transfection of wt-p53 into the anaplastic thyroid cancer cell line FRO reduced c-Abl protein expression. Coincident with these in vitro data, the immunohistochemical study revealed that a high level of c-Abl positive immunostaining was observed in most of the anaplastic carcinoma cases that were strongly positive for p53. These data suggest that p53 status may influence the level of c-Abl protein in thyroid cancer cells.

To clarify the mechanism of cell growth inhibition by SALT I, its effect on the phosphorylation status of c-Abl and ERK1/2, a MAP kinase, was observed. SALT I inhibited the kinase activity of c-Abl in dose- and time-dependent manner in ARO and FRO, but failed to reduce the level of phospho-c-Abl in wt-p53 cell lines. MAP kinase activity was not inhibited by SALT I in any of the cell lines tested. Therefore, drug induced growth suppression in anaplastic cancer cells is not mediated by the “receptor type tyrosine kinase-ras-MAPK” pathway, but is rather associated with inhibition of c-Abl kinase.

Expression of the cyclin-dependent kinase inhibitors, p21^{cip1} and p27^{kip1}, was increased and expression of cyclin A and B1 was decreased in SALT I-treated FRO cells. Similar changes in the expression of p27^{kip1}, cyclin A and B1, and reduction in the expression of cyclin D3 were observed in another anaplastic cancer cell line, ARO, but not in KTC-1 and 1F3 wt-p53 cells. As a consequence, treatment with SALT I induced late S or G2/M transition arrest in the p53-deficient thyroid cell lines. It is worth noting that p21^{cip1} over expression has been linked to S-phase arrest in several other model systems, including p53 null/mutant T98G cells (Potapova et al., J. Biol. Chem. 2000, 275:24767-75). Consistent with our findings, exposure to SALT I increased the mRNA and protein levels of p27^{kip1} in the IL-3 deprived pro-B cell line, BaF3-p210, which overexpresses BCR/ABL (Parada et al., J. Biol. Chem. 2001, 276:23572-80). It is therefore plausible to suggest that inhibition of c-ABL kinase activity by SALT I may cause cell growth inhibition via alteration of the expression/activity of cell cycle modulators. A possible explanation of how SALT I upregulates p21^{cip1} and p27^{kip1} is as follows: c-ABL kinase can phosphorylate PKC-δ leading to an increase of c-Jun NH₂ terminal kinase (JNK) activity (Sun et al., J. Biol. Chem. 2000, 275:7470-3). Our recent work has also shown that the intracellular signaling cascade PKC-δ-MKK7-JNK is activated in ARO cells (Mitsutake et al., Oncogene, 2001, 20:989-96), and JNK specific antisense oligonucleotides have been shown to induce S-phase arrest accompanied by the induction of p21^{cip1}. Therefore, inhibition of the c-ABL-PKC-δ-MKK7-JNK cascade by SALT I may be responsible for the growth inhibition of p53-mutated thyroid cancer cell lines. In conclusion, our results demonstrate that c-Abl is over-expressed in p53 mutated/deficient anaplastic thyroid carcinoma cell lines, and selective inhibition of c-Abl activity by SALT I has a marked cytostatic effect in such cells. Also, SALT I effectively suppresses the in vivo growth of FRO cells implanted into immuno-compromised mice without evident side effects. Thus, use of SALT I is a potential anti-cancer modality for human anaplastic thyroid carcinomas.

### EXAMPLE 2

### Capsules with 4-[(4-methyl-1-piperazin-1-ylmethyl)-N-[4-methyl-3-[[4-(3-pyridinyl)-2-pyrimidinyl]amino]phenyl]benzamide methanesulfonate, β-crystal form

Capsules containing 119.5 mg of SALT I corresponding to 100 mg of COMPOUND I (free base) as active substance are prepared in the following composition:

Composition

| | |
|---|---|
| SALT I | 119.5 mg |
| Cellulose MK GR | 92 mg |
| Crospovidone XL | 15 mg |
| Aerosil 200 | 2 mg |
| Magnesium stearate | 1.5 mg |
| | 230 mg |

The capsules are prepared by mixing the components and filling the mixture into hard gelatin capsules, size 1.

### EXAMPLE 3

### Capsules with 4-[(4methyl-1-piperazin-1-ylmethyl)-N-[4-methyl-3-[[4-(3-pyridinyl)-2-pyrimidinyl]amino]phenyl]benzamide methanesulfonate, □-crystal form

Capsules containing 119.5 mg of SALT I corresponding to 100 mg of COMPOUND I (free base) as active substance are prepared in the following composition:

Composition

| | |
|---|---|
| Active substance | 119.5 mg |
| Avicel | 200 mg |
| PVPPXL | 15 mg |
| Aerosil | 2 mg |
| Magnesium stearate | 1.5 mg |
| | 338.0 mg |

The capsules are prepared by mixing the components and filling the mixture into hard gelatin capsules, size 1.

### EXAMPLE 4

### Protocol for the Clinical Study of SALT I in the Treatment of Refractory Progressive Thyroid Carcinoma

A—Study subjects—Inclusion criteria
1) Have thyroid carcinoma with a metastatic lesion and performance status of 0 to 2 (See Table below); and, in principle, have clinical aggravation of disease suggestive of undifferentiated transformation from papillary or follicular carcinoma, which does not or is unlikely to respond to other treatments.
2) Aged between 20 and 80 years.
3) Have a lesion that is evaluable by CT/MRI imaging and tumor marker (thyroglobulin) prior to start of treatment.
4) Have a certain extent of cardiac, pulmonary and renal function and has no serious bleeding tendency, i.e., fulfill the following criteria in principle:
   a) Ejection fraction >50% on ultrasonic cardiography, with no history of ischemic heart disease during one year preceding study entry.
   b) pO₂>60 mmHg in blood gas analysis, regardless of presence or absence of lung metastasis.
   c) Serum bilirubin <2 mg/dL, regardless of presence or absence of liver metastasis.
   d) Serum creatinine <2 mg/dL.
   e) Leukocytes ≧2000/mm³ and platelets ≧50000/mm³.
   f) PT>50%, APTR<50 sec, Fbg>100 mg/dL, and FDP<20 μg/mL.
5) Have no active infection difficult to control.

6) Consent to take part in this study and give written informed consent.

Performance Status (PS) (by SWOG)

| Grade | Performance Status |
|---|---|
| 0 | No symptoms. Able to carry out all normal social activity |
| | without restriction; able to act in the same way as before |
| | the occurrence of the disease. |
| 1 | Slight symptoms. Restricted in physically strenuous activity, |
| | but ambulatory and able to carry out light work (e.g. housework, |
| | office work). |
| 2 | Ambulatory and capable of self-care, sometimes with the need of |
| | a little assistance; unable to carry out any work; up and about |
| | more than 50% of waking hours. |
| 3 | Capable of only limited self-care, sometimes with the need of |
| | assistance; confirmed to bed more than 50% of waking hours. |
| 4 | Completely disabled; cannot carry on any self-care; need of |
| | complete assistance; totally confirmed to bed. |

These criteria are the index of performance status. When activities are restricted in local areas, it will be clinically evaluated.

B—Methods of the Study

This clinical study is part of the Phase I/II clinical study which is exploratory in nature.

1. Treatment Schedule

SALT I is administered at a dose corresponding to 400 mg of COMPOUND I after a meal once daily. If it is effective and cause no adverse effects or only mild acceptable adverse effects, SALT I treatment is continued for a maximum of six months. When any mild adverse effect occurs, the dose is decreased to 200 mg once daily depending on the degree of the adverse effect. At two months of treatment, the effectiveness of SALT I is assessed and subsequent treatment decisions (continuation of treatment, dose increase, discontinuation of treatment, etc.) made. In principle, where the tumor size is reduced by half, the dose is increased up to 800 mg/day and continued for a maximum of six months. Where the tumor size is larger at two months of treatment, or where the tumor size remains unchanged but any further improvement in tumor associated symptoms is preferred, the conduct of radiotherapy in combination with SALT I treatment is considered in cases where it is possible. No concomitant use of an anticancer drug is permitted in principle. Drugs that relieve other conditions or symptoms may be used concomitantly with caution exercised for their adverse effects.

2. Assessment of Effectiveness

The effectiveness of SALT I is assessed at 1, 2, 3, 4, 5 and 6 months of treatment on the basis of (1) improvement of the condition as determined by physiological findings and the change in consistency of tumor, (2) the tumor size determined by imaging; and (3) the change in tumor marker.

Assessment Items

(1) Background factors of subjects: Medical Record No., ID No., initials, sex, date of birth, height, body weight; complications, previous illnesses, present illnesses, previous treatment, family history

(2) Dosage of COMPOUND I

(3) Records of compliance and concomitant medication

(4) Subjective symptoms and objective findings

(5) Blood pressure and pulse rate

(6) Hematology (red blood cells, white blood cells, platelets, differential WBC): twice monthly

(7) Biochemistry (liver function, renal function, blood glucose, LDH, CPK): twice monthly

(8) Tumor marker (thyroglobulin): once monthly

(9) Tumor-occupying site, tumor size, and extent of infiltration on CT or MRI imaging: Once every two months

(10) Chest x-ray (front): Once every two months

(11) Check of tumor associated symptoms

(12) Urinalysis

C-Preliminary Results

First patient entry into clinical study in December 2002. SALT I treatment was started without any other therapy. Tumors have stopped their growth for 3 months in contrast with rapid growth and invasion during the previous year.

1. A method consisting of 4-(4-methylpiperazin-1-ylmethyl)-N-[4-methyl-3-(4-pyridin-3-yl)pyrimidin-2-ylamino)phenyl]-benzamide of formula I 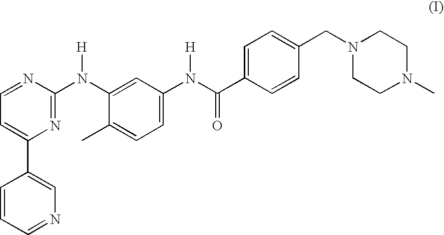
or a pharmaceutically acceptable salt thereof for the manufacture of a medicament for the treatment of anaplastic thyroid carcinomas.

2. A method consisting of 4-(4-methylpiperazin-1-ylmethyl)-N-[4-methyl-3-(4-pyridin-3-yl)pyrimidin-2-ylamino)phenyl]-benzamide of formula I or a pharmaceutically acceptable salt thereof in the treatment of anaplastic thyroid carcinomas.

3. A method of treating humans suffering from anaplastic thyroid carcinoma which comprises administering to a said human in need of such treatment a dose, effective against said disease, of 4-(4-methylpiperazin-1-ylmethyl)-N-[4-methyl-3-(4-pyridin-3-yl)pyrimidin-2-ylamino)phenyl]-benzamide or a pharmaceutically acceptable salt thereof.

4. A method according to claim 3 wherein a daily dose of 50 to 600 mg of 4-(4-methylpiperazin-1-ylmethyl)-N-[4-methyl-3-(4-pyridin-3-yl)pyrimidin-2-ylamino)phenyl]-benzamide of formula I is administered to an adult.

5. A method according to claim 3 wherein the monomethanesulfonate salt of 4-(4-methylpiperazin-1-ylmethyl)-N-[4-methyl-3-(4-pyridin-3-yl)pyrimidin-2-ylamino)phenyl]benzamide of formula I is administered.

6. A method according to claim 3 which comprises administering a pharmaceutically effective amount of 4-(4-methylpiperazin-1-ylmethyl)-N-[4-methyl-3-(4-pyridin-3-yl)pyrimidin-2-ylamino)phenyl]-benzamide of formula I or a pharmaceutically acceptable salt thereof to the human subject once daily for a period exceeding 3 months.

7. A method according to claim 1 wherein 4-(4-methylpiperazin-1-ylmethyl)-N-[4-methyl-3-(4-pyridin-3-yl)pyrimidin-2-yl-amino)phenyl]-benzamide of formula I is in mesylate salt form and in the beta crystal form thereof.

8. A method according to claim 1 wherein the anaplastic thyroid carcinoma is harboring mutated p53.
